(19) United States
(12) Patent Application Publication
(10) Pub. No.: US 2020/0046337 A1
(43) Pub. Date: Feb. 13, 2020

(54) MONOFILAMENT SUTURE ANCHOR

(71) Applicant: ARTHREX, INC., Naples, FL (US)

(72) Inventor: Peter J. Dreyfuss, Naples, FL (US)

(21) Appl. No.: 16/102,001

(22) Filed: Aug. 13, 2018

(51) Int. Cl. A61B 17/04; A61F 2/08

(52) U.S. Cl. CPC A61B 17/0401; A61F 2/0811; A61B 2017/0464; A61B 2017/0414; A61F 2002/0817; A61F 2002/0852; A61B 2017/0412

(57) ABSTRACT

Knotless surgical constructs and methods of tissue repairs. A monofilament suture is formed and then fused to obtain a fused monofilament suture anchor. The monofilament suture anchor has a design with barbs for fixation, hoop geometry for fixation bias, as well as a head design for tissue fixation and compression that has no points which may cause irritation. The monofilament suture anchor has a simple and compact design that can be easily scaled based on

### BACKGROUND

The disclosure relates to surgical devices and, more specifically, to soft suture anchor constructs and associated methods of tissue repairs.

### SUMMARY

Soft anchor constructs and methods of tissue repairs are disclosed. A soft anchor construct can create a knotless repair. A monofilament suture is formed and then fused/bonded to obtain a fused monofilament suture anchor. The monofilament suture may be welded to form a welded monofilament suture anchor. The monofilament suture anchor has a design with barbs for fixation, hoop geometry for fixation bias, as well as a head design for tissue fixation and compression that has no points which may cause irritation. A soft anchor construct can be knotless. A knotless soft anchor construct has a simple and compact design that can be easily scaled based on need.

Methods of tissue repair are also disclosed. A first tissue is approximated to a second tissue with a knotless surgical construct that includes a monofilament suture anchor. In an embodiment, a knotless monofilament suture anchor includes a monofilament suture that is fused/joined/welded/bonded at different points or regions or segments along its length. In an embodiment, a monofilament suture anchor is attached to a first tissue and to a second tissue. In an embodiment, a monofilament suture anchor attaches first tissue to second tissue. The monofilament suture anchor offers a simple mechanism to insert and fix tissue with minimal implant bulk. The monofilament suture anchor is knotless and self-inserting, with no additional steps needed from surgeon.

### BRIEF DESCRIPTION OF THE DRAWINGS

FIG. 1 illustrates a surgical construct according to an exemplary embodiment.

FIG. 2 illustrates a surgical construct according to another exemplary embodiment.

FIG. 3-5 illustrate exemplary methods of tissue repair with surgical constructs.

### DETAILED DESCRIPTION

The disclosure provides surgical constructs for securing soft tissue to bone with a knotless construct. The knotless construct is a soft anchor construct (a suture anchor) that includes a monofilament which is joined/fused/welded/bonded/connected at different points/regions/segments to form a fused monofilament suture anchor. The monofilament suture anchor may be welded by ultrasonic welding.

In an embodiment, a surgical construct is a knotless multi-loop construct. The construct is a soft anchor construct in the form of a monofilament suture that is joined/fused/welded/bonded/connected to obtain a joined/fused/welded/bonded/connected monofilament suture anchor. The monofilament suture anchor has a design with barbs for fixation and hoop geometry for fixation bias, as well as a head design for tissue fixation and compression that has no points which may cause irritation.

The monofilament suture anchor provides a simple mechanism to insert and fix tissue with minimal implant bulk. The anchor is welded to provide barbs for tissue/bone fixation and a “hat” for tissue compression.

An exemplary embodiment has the ends fixed in the middle part of the anchor construct. In another embodiment, the ends of the monofilament are overlapped and useful as barbs for fixation. The dumbbell-shaped end is what fixes on top of tissue. The big loop end is driven into tissue by its middle region (which pulls the whole construct behind it in). Once the construct is in tissue, it wants to return to its big loop shape and that bias provides the fixation (barbs, and/or other features).

The monofilament suture anchor is knotless and self-inserting with no additional steps needed from surgeon. The simple and compact design can be scaled based on need.

Methods of tissue repair are also disclosed. In an embodiment, a monofilament suture anchor can attach a first tissue to a second tissue, for example, soft tissue to bone, or soft tissue to soft tissue. The monofilament suture anchor can be passed by conducting a single pass through or around tissue to be repaired (ligament, tendon, graft, etc.) and then inserted through a second tissue, for example, bone. At least one loop of the monofilament suture anchor may rest upon/within a first tissue and at least another loop of the monofilament suture anchor may rest upon/within a second tissue, to attach the first tissue to the second tissue. The first tissue may be similar to or different from the second tissue.

A method of knotless tissue repair comprises inter alia providing a soft suture anchor where a first loop of a monofilament suture anchor is fixed in a first tissue and a second loop of the monofilament suture anchor is fixed in a second tissue, all steps being conducted in a knotless manner.

In an embodiment, a method of knotless tissue repair is conducted by inter alia inserting a surgical construct in a first tissue and a second tissue, the surgical construct including a monofilament suture anchor in the form of a flexible member (flexible strand/material/elongated member); and securing the flexible member in a knotless manner to both the first tissue and the second tissue. The method may further comprise the steps of securing at least a first fused loop of the monofilament suture anchor to the first tissue, and securing at least a second loop of the monofilament suture anchor to the second tissue. The first tissue may be similar to or different from the second tissue. The monofilament suture anchor may include a plurality of fused loops, which may be different and/or similar. The monofilament suture anchor may consist essentially of a monofilament suture.

The surgical constructs and methods of the present disclosure provide apparatus and methods for tissue repair, for example, soft tissue to bone repair, or soft tissue to soft tissue repair. The surgical constructs allow for simplified repairs, and help simplify shuttling and managing of suture in a tissue repair such as rotator cuff repair.

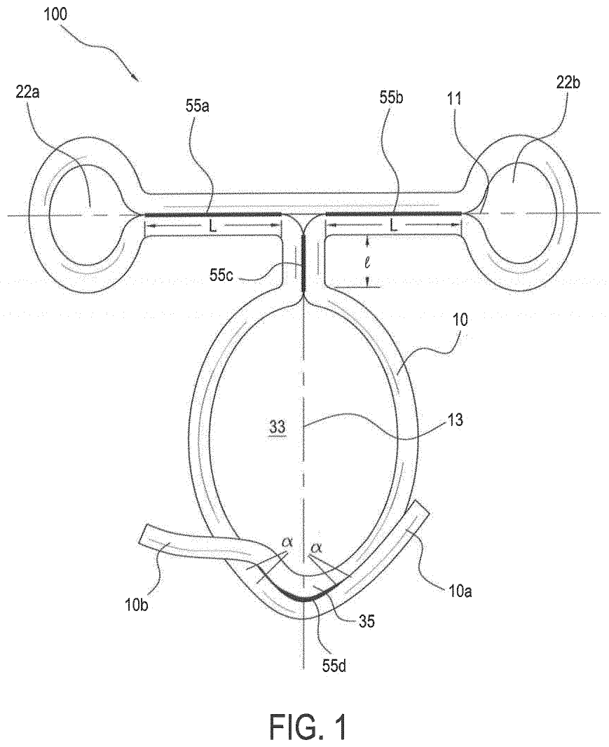

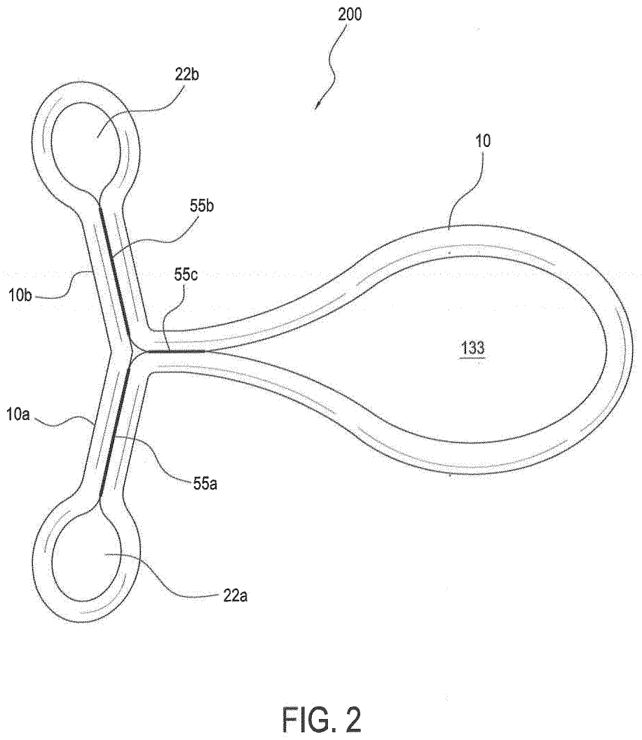

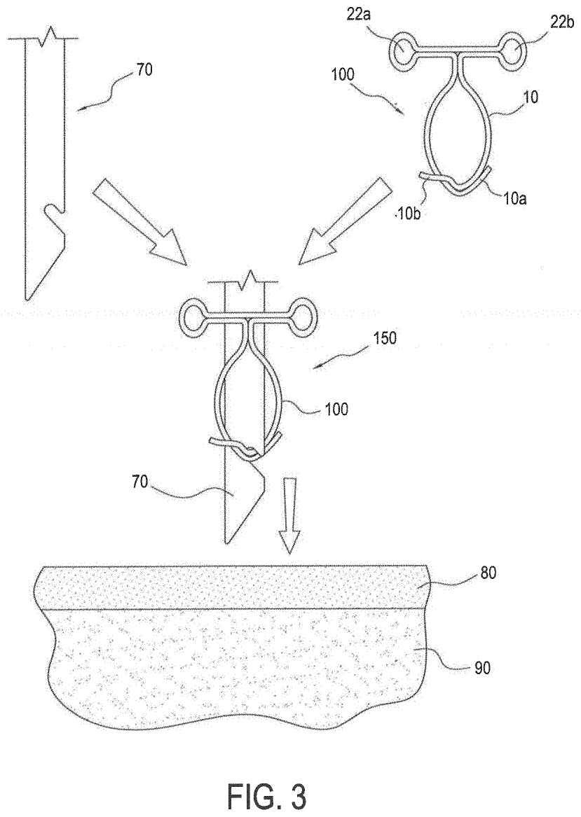

Referring now to the drawings, where like elements are designated by like reference numerals, FIGS. 1 and 2 illustrate structural components of surgical construct 100, 200 which is a monofilament suture anchor 100, 200 (knotless soft anchor construct 100, 200; construct 100, 200; knotless surgical construct 100, 200) of the present disclosure. FIGS. 3-5 illustrate steps of methods of tissue repair 101 with exemplary surgical construct 100.

FIG. 1 illustrates exemplary monofilament suture anchor 100 which is a multi-loop construct. FIG. 2 illustrates another exemplary surgical construct 200 which is also a monofilament suture anchor with a multi-loop flexible knotless construct with a slightly different design and with a different number of joined/fused/welded/bonded/connected regions or segments.

Surgical construct 100 is a flexible and knotless construct which includes flexible strand 10 which is in the form of a single elongated member, fiber, or material 10. Flexible strand 10 is a monofilament, for example, a monofilament suture which may contain a polymer or a combination of polymers. In an embodiment, flexible strand 10 is formed essentially of one strand of surgical suture material, for example, a thermoplastic polymer, that is amenable to joining, fusion, welding or similar bonding/fusion techniques. Suitable materials for the flexible strand 10 are polymers, especially thermoplastic materials such as, for example, nylon (polyamide, PA), polypropylene, Dacron® (a suitable type of polyester), polyglycolic acid (PGA), polyglyconate, and polydioxanone. The flexible strand 10 can include a single filament, or fiber, or can include multiple continuous filaments, segments or regions of filaments that have different configurations (for example, different diameters and/or different compositions, as long as the strand remains a monofilament). The filament regions/segments may each be homogenous (i.e., formed of a same material) or may be a combination of homogenous and heterogenous (i.e., formed of a plurality of materials).

The monofilament 10 may be non-absorbable and/or formed of absorbable and non-absorbable combinations. Exemplary non-absorbable materials may include silk, cotton, rubber, nylon, polypropylene, polyethylene, ultrahigh molecular weight polyethylene (UHMWPE), polyethylene terephthalate (PET), and polyesters and copolymers thereof, or combinations thereof. The monofilament 10 may be a cord having a circular cross-section; however, the disclosure contemplates other cross-sections such as elliptical, rectangular, etc. depending on the intended application. In addition, the monofilament may have regions, segments or lengths of various diameters throughout its length.

In the exemplary embodiment of FIG. 1, surgical construct 100 includes monofilament suture 10 in the form of single flexible strand; however, the disclosure contemplates embodiments wherein the monofilament suture anchor is formed of a plurality of monofilaments, which may have similar or different characteristics, sizes, shapes, properties, etc., depending on the intended application.

As shown in FIG. 1, monofilament suture 10 is shaped/formed to have the specific design shown thereof, i.e., with two smaller loops 22a, 22b and a larger loop 33. Loops 22a, 22b, 33 are closed, flexible loops having a general circular or oval configuration; however, loops 22a, 22b, 33 may have any other desired configurations, for example, elliptical among many others. The lengths and/or diameter of loops 22a, 22b, 33 may vary depending on the application. For example, loops 22a and 22b may be about similar in size (length and/or diameter) whereas loop 33 is different from loops 22a, 22b. As shown in FIG. 1, loop 33 forms a narrow part or angulation 35 in the form of a barb, which helps with tissue fixation, as detailed below.

Loops 22a, 22b, 33 are formed by bonding, fusing, welding and/or any joining process of various segments or regions of the monofilament 10, for example, by a welding process. Initially, two terminal ends 10a, 10b of monofilament suture 10 are brought together to overlap as shown in FIG. 1. Segments of monofilament 10 are then joined together under energy such as heat until the segments fuse together and form loops 22a, 22b, 33. FIG. 1 illustrates the construct 100 with four exemplary joined/fused/welded/bonded/connected regions 55a, 55b, 55c, 55d; however, the disclosure contemplates any number of joined/fused/welded/bonded/connected regions that allow the formation of at least two loops (which may be different or similar) to allow fixation on a first tissue and a second tissue, respectively.

Various types of welded segments and exemplary loops 22a, 22b, 33 can be formed by the application of, for example, ultrasonic, thermal, laser, electrical arc discharge, or thermal energy to the segments, which can be-joined, for example, in an overlapped joint. In an exemplary embodiment, welding of the segments is accomplished by the application of ultrasonic energy. In such case, when the energy is applied to two or more touching segments of monofilament 10, the segments melt and join together to form a plurality of welding regions such as welding regions 55a, 55b, 55c, 55d extending along the monofilament 10. FIG. 1 illustrates four exemplary welding regions 55a, 55b, 55c, 55d that form three exemplary loops 22a, 22b, 33.

FIG. 1 illustrates exemplary fused loops 22a, 22b, 33 positioned in a particular orientation with respect to longitudinal axis 11 and transversal axis 13 of monofilament suture anchor 100. For example, loops 22a, 22b may extend in a first direction and loop 33 may extend in a second direction, which may be similar or different from the first direction. Similarly, segments 55a, 55b, 55c, 55d may all extend along various directions and orientations and may form various angles relative to each other (or combinations thereof). In the exemplary embodiment of FIG. 1, loops 22a, 22b as well as joined/fused/welded/bonded/connected regions 55a, 55b extend along the longitudinal axis 11 of the anchor (i.e., along a first direction which is about parallel to the longitudinal axis 11). Loop 33 and joined/fused/welded/bonded/connected region 55c extend along the transversal axis 13 of the anchor (i.e., along a second direction which is about parallel to the transversal axis 13). Joined/fused/welded/bonded/connected region 55d of FIG. 1 extends at an angle α relative to the transversal axis 13 (on each side of the axis).

FIG. 2 illustrates another exemplary surgical construct 200 which is also a monofilament suture anchor 200 (knotless soft anchor construct 200; construct 200; knotless surgical construct 200) of the present disclosure. Surgical construct 200 of FIG. 2 is similar to surgical construct 100 of FIG. 1 but differs in that the two ends 10a, 10b of monofilament 10 are not brought together to overlap and form loop 33 (shown in FIG. 1) but rather are fused/bonded/joined/welded in regions 55a, 55b, for example. Loop 133 is larger than loops 22a, 22b and the construct 200 includes only three joined/fused/welded/bonded regions 55a, 55b, 55c.

The surgical constructs 100, 200 create knotless repairs wherein the fused loops 22a, 22b, 33, 133 are passed through first and second tissues and then fixated at various locations on the first and second tissues.

FIGS. 3-5 illustrate exemplary steps of a method of knotless tissue repair with monofilament suture anchor 100 of FIG. 1. The method is conducted by securing monofilament suture anchor 100 to an inserter 70 to form assembly 150 (FIG. 3) and providing the assembly 150 in the vicinity of first tissue 80 and second tissue 90, as shown in FIG. 3.

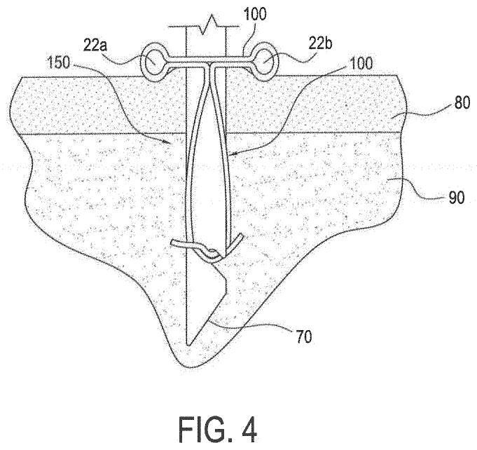

The monofilament suture anchor 100 is inserted or passed through the first and second tissues 80, 90 as shown in FIG. 4. Depending on application, a hole may be formed in one or both of the first and second tissues (for example, a hole may be formed in bone 90).

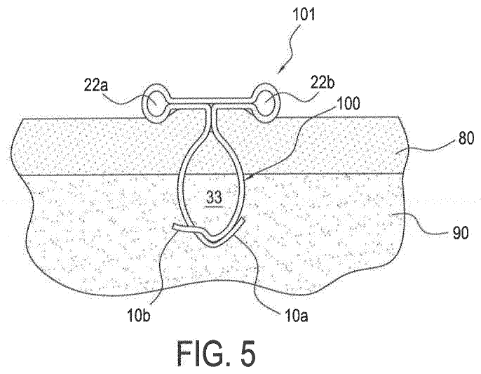

Inserter 70 is removed (FIG. 5) to obtain repair 101 with loops 22a, 22b positioned over and in contact with first tissue 80 (in a dumbbell configuration that provides a “hat” like design), and larger loop 33 positioned within the second tissue 90 with ends 10a, 10b acting as barbs for fixation bias. The method provides a simple mechanism to insert the monofilament suture anchor 100 in a knotless manner, and to fix tissue with minimal implant bulk. Tissue compression is achieved with no knots and points that could cause irritation of tissue and final repair. The monofilament suture anchor 100 is knotless and self-inserting and does not require any other additional steps from surgeon. The design of the monofilament suture anchor 100 is compact and simple and can be easily scaled based on need.

Flexible strand 10 may be made of any known monofilament fiber such as monofilament suture, and portions of it may include fibers of ultrahigh molecular weight polyethylene (UHMWPE) or the FiberWire® suture (disclosed in U.S. Pat. No. 6,716,234, the disclosure of which is hereby incorporated by reference in its entirety herein). FiberWire® suture is formed of an advanced, high-strength fiber material, namely ultrahigh molecular weight polyethylene (UHMWPE), sold under the tradenames Spectra (Honeywell) and Dyneema (DSM), braided with at least one other fiber, natural or synthetic, to form lengths of suture material. The preferred FiberWire® suture includes a core within a hollow braided construct, the core being a twisted yarn of UHMWPE.

The monofilament may be also formed of a stiff material, or combination of stiff and flexible materials, depending on the intended application. The monofilament may be also coated and/or provided in different colors. The knotless anchors of the present disclosure can be used with any type of flexible material or suture that forms a loop and that is amenable to fusion or welding or similar processes.

Mono filament suture anchors 100, 200 may be preferably coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the suture, loop security, pliability, handleability or abrasion resistance, for example.

The monofilament 10 of the surgical constructs 100, 200 may be also provided with tinted tracing strands, or otherwise contrast visually with other areas/regions of the construct, which remains a plain, solid color, or displays a different tracing pattern, for example. Various structural elements of surgical constructs 100, 200 may be visually coded, making identification and handling of the suture loops and ends simpler. Easy identification of suture in situ is advantageous in surgical procedures, particularly during arthroscopic surgeries, such as endoscopy and laparoscopy.

The surgical construct 100, 200 of the present disclosure has applicability to suture applications that may be employed in surgical procedures such as rotator cuff repair, Achilles tendon repair, patellar tendon repair, ACL/PCL reconstruction, hip and shoulder reconstruction procedures, and applications for suture used in or with suture anchors.

An exemplary method of tissue repair may comprise inter alia the steps of inserting a monofilament suture anchor 100, 200 with a plurality of fused suture loops through first and second tissues; and securing at least one of the plurality of fused suture loops to one of the first and second tissues, and securing at least another of the plurality of fused suture loops to the other of the first and second tissues, in a knotless manner.

In an embodiment, a method of knotless tissue repair is conducted by inter alia inserting a surgical construct 100, 200 through soft tissue 80 and bone 90, the surgical construct including a monofilament suture 10 in the form of a flexible construct with a first plurality of fused loops 22a, 22b and a second plurality of fused loops 33, 133; securing the first plurality of fused loops 22a, 22b to soft tissue 80 and securing the second plurality of fused loops 33, 133 to bone 90, in a knotless manner.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

1. A monofilament suture anchor comprising:
a monofilament suture with a first end and a second end, two closed fused loops extending in a first direction, and another closed fused loop extending in a second direction, wherein the first direction is about perpendicular to the second direction, and wherein the two closed fused loops and the another closed fused loops are formed by joining together segments of the monofilament suture, and wherein each of the two closed fused loops and the another closed fused loop defines an opening which is circular or oval.

2. (canceled)

3. (canceled)

4. The monofilament suture anchor of claim 1, wherein the two closed fused loops and the another closed fused loop are formed by ultrasonically welding segments of the monofilament suture.

5. The monofilament suture anchor of claim 1, wherein the monofilament suture is formed of a polymer.

6. The monofilament suture anchor of claim 1, wherein the first end and the second end overlap each other and are joined together to form the another closed fused loop extending in the second direction.

7. (canceled)

8. The monofilament suture anchor of claim 1, wherein the two closed fused loops extending in the first direction are configured to be secured to a first tissue, and the another closed fused loop extending in the second direction is configured to be secured to a second tissue.

9. The monofilament suture anchor of claim 8, wherein the first tissue is soft tissue and the second tissue is bone.

10-15. (canceled)
